(19) United States
(12) Patent Application Publication
(10) Pub. No.: US 2011/0301483 A1
(43) Pub. Date: Dec. 8, 2011

(54) LOCAL LUNG MEASUREMENT AND TREATMENT

(75) Inventors: Niyazi Beyhan, Santa Clara, CA (US); Srikanth Radhakrishnan, Cupertino, CA (US)

(73) Assignee: Pulmonx Corporation, Redwood City, CA (US)

(21) Appl. No.: 12/963,412

(22) Filed: Dec. 8, 2010

Related U.S. Application Data
(60) Provisional application No. 61/289,848, filed on Dec. 23, 2009.

(51) Int. Cl. A61B 5/097; A61B 5/091; A61B 5/08

(52) U.S. Cl. 600/532; 600/529; 600/538

(57) ABSTRACT

A method of determining potential treatment sites in a diseased lung is disclosed, in which an assessment catheter is introduced into a lung passageway. The catheter has a distal portion comprising an occluding member and a proximal portion configured to operatively mate with an external console. The catheter is used to identify one or more assessment sites within the airways of the lung. At each assessment site, at least one physiological, anatomical or biological characteristic is determined. A characteristic score for each assessment site is calculated based on a predetermined algorithm; and a treatment is determined based on the scores of the assessment sites. The algorithm takes into account several parameters including the disease characteristics as well as the number and proximity of each assessment site to at least one of the diseased regions. The method envisages treatment of emphysema, asthma or bronchopleural leak.

### CROSS-REFERENCE TO RELATED APPLICATION

This application claims the benefit of Provisional Application No. 61/289,848 (Attorney Docket No. 017534-007600US), filed on Dec. 23, 2009, the full disclosure of which is incorporated herein by reference.

### BACKGROUND OF THE INVENTION

1. Field of the Invention

This invention relates generally to measurement of physical, chemical and anatomic parameters in the lung for diagnosis of pulmonary disease and localized treatment of the disease.

2. Description of the Related Art

Chronic obstructive pulmonary disease (COPD) is a significant medical problem affecting 16 million people, or about 6% of the U.S. population. Specific diseases in this group include chronic bronchitis, asthmatic bronchitis, and emphysema. Lung cancer, as another example, is among the most prevalent forms of cancer, and causes more than 150,000 deaths per year in the U.S. Many methods have been proposed and are in use for diagnosis and treatment in the advanced stages of disease progression. These stages are marked by significant damage to the lung tissue so that the difference between healthy and diseased tissue is readily apparent during the diagnosis. Typically, imaging tests such as chest x-rays, computed tomography (CT) scans, Magnetic Resonance Imaging (MRI), perfusion scans, and bronchograms provide a good indicator of the location, homogeneity and progression of the diseased tissue. However, these tests provide a diagnosis from the global (i.e., whole lung) level, rather than from the local (i.e., from the lobar or segmental) level.

Recently, the trend has been toward early diagnosis of disease conditions using a variety of new techniques. Early diagnosis of lung disease has many benefits such as increased patient wellbeing along with reduced morbidity, lowering of treatment costs, and decreased load on the health care system. Such diagnosis could depend on the identification of markers or indicators of the disease condition (Lacoma et al., Eur. Respir. Rev. 2009; 18: 112, 96-104). Biochemical markers such as nitric oxide (Brindicci et al., Eur. Respir. J 2005; 26:52-59) or peroxynitrite (Osoata et al., Chest June 2009, 135(6): 1513-1520) measured in exhaled air have been used to characterize COPD for many years, although local measurement within the lung has not been reported. The markers of disease could also be anatomical changes such as constriction of airways or tearing of alveoli, or functional changes such as changes to blood flow or air flow, all of which are local indications of disease.

Functional tests could provide good physiological indicators of disease progression. Functional testing, such as spirometry, plethysmography, oxygen saturation, and oxygen consumption stress testing, among others, is being used of late to determine the course of treatment for the patient. However, identification of appropriate markers in functional and physiological testing is difficult (Jones and Agusti, Eur Respir J 2006; 27: 822-832). Moreover, since these tests are also largely global, locating the specific, local areas of disease damage where treatment is required is challenging. Interventional measurements locally within the lung would prove more beneficial.

Some methods and devices for localized diagnosis and functional testing to identify specific areas of disease within the lung are disclosed in copending U.S. Published Patent Applications 2007/0142742, 2008/0249503 and 2008/0200797, which are incorporated herein by reference. These applications discuss the measurement of collateral ventilation at the lobar and segmental levels in patients with emphysema. The measurement of collateral ventilation is done in a minimally invasive manner by occluding the airway and determining the change in pressure and/or measuring the composition of the gas within the lung compartment. The measurements may then be followed by an appropriate treatment to effect lung volume reduction.

Measurement of collateral ventilation through the use of external pressurization is disclosed in U.S. Pat. No. 6,692,494 to Cooper et al. Disadvantages of such a technique include the possibility of additional damage to lung tissue already weakened by disease.

The use of local anatomical changes for localized treatment in asthmatic lungs is disclosed in U.S. Published Patent Application No. 2006/0254600 to Danek et al. This reference describes the measurement of several parameters such as airway diameter, airway compliance, airway inflammation, etc., that are indicative of asthma. Some of these parameters are measured after artificial stimulation by introducing an agent at the airway location, which is specific to asthma treatment. Though this reference also discusses measurement of local changes in pressure for determining the course of treatment, the specific details of the measurement technique are not disclosed.

The use of chemical markers for diagnosing lung disease is disclosed in U.S. Published Patent Applications 2006/0074282 to Ward et al. and 2007/0261472 to Flaherty et al. The 2006/0074282 reference discloses the use of Raman spectroscopy to detect biochemical markers at locations within the lung through a flexible optical conduit, or externally, in exhaled air. The biomarkers include those relevant to lung disease such as nitric oxide-hemoglobin complex. However, there is no description of a specific device used for such measurement and no localized treatment is disclosed. The 2007/0261472 reference discloses non-invasive sensing of nitric oxide in exhaled air for diagnosis of asthma-related hypoxia. However, the method uses global measurement in exhaled air and the affected portion of lung cannot be identified.

Markers and indicators of respiratory diseases can be quite complex, as there may be no universal biochemical marker or level of indication that is applicable for diagnosis of diseases such as emphysema or asthma. Clinical studies show that levels of the most commonly used biomarkers must be individualized and their changes monitored for deriving meaningful conclusions. Physiological and anatomical indications may also be required to be monitored along with biochemical markers for identifying the areas most severely affected by disease. Because of the large number of variables and the absence of unique determinants, it is not feasible for a physician to merely study the data and decide on locations requiring treatment. Partly addressing this shortcoming, U.S. Pat. No. 7,517,320 to Wibowo et al. discloses a method of using imaging data from emphysematous lungs to obtain a ranking of tissue regions for treatment. The ranking is based on parameters such as airway diameter, airway thickness, collateral ventilation, degree of tissue destruction, etc. However, these parameters are obtained only by analyzing image data and not by local measurement. Given that pulmonary disease indicators can be complex, a sophisticated approach toward multiparametric analysis of the clinical data would be more effective for diagnosis and treatment.

If diagnoses are not localized, the corresponding interventional treatments involving therapeutic agents may cause side effects that are detrimental. For example, steroids may be administered to a patient by inhalation to control asthma or emphysema. However, the dosage required for inhalation treatment is much higher than that required to locally treat an airway. In inhalation therapy, to ensure that the treatment is effective, a high concentration of the drug must be used, and the whole lung must be treated. A high proportion of the ingested drug is ineffective and simply passes through the system, producing a variety of harmful side effects due to reaction with non-diseased portions of the lung and body. Localized treatment, while being highly effective (because it introduces treatment exactly where it is needed), reduces global or systemic intake and minimizes side effects.

Some devices have sought to address this shortcoming by using localized treatment. For example, U.S. Published Patent Application 2008/0200797 (cited above) also discloses the implantation of a one-way valve in the airway at an appropriate location for effecting gradual lung volume reduction. U.S. Published Patent Application 2008/0249503 (also cited above) further discloses the use of therapeutic agents for treatment. These treatments would be enhanced by the provision of ameliorated diagnostic methods at the localized level.

The above discussion of the prior art shows that there is a need for a system that provides for local diagnosis of various parameters within a diseased lung, a better way of ranking various sites based on locally measured parameters, and a way to treat locations that are most affected by disease, in a comprehensive manner. At least some of these objectives are met by the embodiments described below.

### BRIEF SUMMARY OF THE INVENTION

In one aspect of the present invention, a method is described for selecting one or more treatment sites in a diseased lung. In one embodiment, the method may involve: introducing an assessment catheter into an airway leading to a first assessment site in the lung; expanding an occluding member on the catheter to form a seal with an inner wall of the airway and thus isolate the first assessment site; measuring at least one physiological, anatomical or biological characteristic of the first assessment site using the catheter; calculating a score for the first assessment site based on the measured characteristic and a predetermined algorithm; repeating the steps for at least a second assessment site in the lung; and selecting at least one treatment site based on the scores of the assessment sites. In various embodiments, the method may further include repeating the steps for at least a third assessment site, fourth assessment site, etc.

In some embodiments, at least the calculating step may be performed by a console coupled with a proximal end of the catheter, and the scores may be displayed on the console. In some embodiments, the physiological characteristic is collateral ventilation. For example, the collateral ventilation may be assessed to treat an air leak or may be assessed to select an appropriate lung segment for treatment of emphysema. In one embodiment, the biological characteristic may be nitric oxide. In one embodiment, the anatomical characteristic may be an air leak. In some embodiments, the algorithm may be based on a determined number of diseased regions in the lung, the at least one physiological, anatomical or biological characteristic of each site, and proximity of each assessment site to at least one of the diseased regions.

In some embodiments, the method may further involve treating the treatment site(s). Optionally, the method may further include introducing an assessment catheter into the lung to confirm efficacy of treatment. In one embodiment, treating the site may involve implanting a one-way flow control element into an airway leading to a portion of the lung afflicted by emphysema. In various embodiments, the flow control element may include but is not limited to a plug, a one-way valve or a two way valve. In some embodiments, the flow control element is provided with a drug depot configured to provide sustained release of a drug. For example, the drug depot may be configured to release one or more steroids and/or anticholinergics.

In other embodiments, treating may involve performing endoscopic lung volume reduction. Alternatively, treating may involve introducing a drug into the treatment site through a treatment catheter. In yet other embodiments, treating may involve performing bronchial thermoplasty. Alternatively, treating may involve installation of a chest tube.

In another aspect, a method for assessing the effectiveness of a treatment may involve identifying an airway that has been occluded with a one-way valve, where the one-way valve is configured to allow expiration but limit inhalation. The method may further include introducing a catheter into the identified airway, the catheter including a distal end, a proximal end, and a lumen therebetween. The distal end of the catheter may include an expandable occluding element configured to sealingly engage the airway, and the proximal end may include an inflation port to expand the occluding element. The lumen may be in-line with at least one sensor for measuring a respiratory characteristic. The method may further involve measuring flow through the airway to determine whether flow exists during inhalation, where the presence of flow indicates ineffective valve placement. Optionally, the method may further involve measuring pressure during inhalation, where the presence of pressure indicates ineffective valve placement.

The methods and devices described herein may be useful in diagnosis and/or treatment of diseased lungs afflicted with emphysema, cancer, asthma, air leak, or any of a number of other lung ailments.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1A shows a diagram of a catheter for local measurement of a lung parameter in accordance with an embodiment of the present invention.

FIGS. 1B, 1C and 1D show embodiments of the isolation catheter in which the sampling lumen is configured to have a continuous or discontinuous variation in diameter.

FIG. 2 shows the measurement catheter accessing a target lung compartment for measurement.

FIG. 3 shows a diagram of a console in accordance with an embodiment of the present invention.

FIG. 4 shows a schematic flow diagram of the method of the present invention.

FIGS. 5A to 5C show installation of a flow control element to effect lung volume reduction.

FIGS. 5D and 5E show use of a flow control element with a drug depot for treatment.

FIG. 6 shows the detection of a post-treatment air leak by the catheter.

### DETAILED DESCRIPTION OF THE INVENTION

Although the detailed description contains many specifics, these should not be construed as limiting the scope of the invention but merely as illustrating different embodiments. Thus, the scope of the invention may include other embodiments not discussed in detail. Various other modifications, changes and variations may be made in the arrangement, operation and details of the methods and systems of the embodiments disclosed herein without departing from the spirit and scope of the invention as described.

Methods for treating lung disease according to some embodiments may involve inserting a catheter into the lung to make local measurements of one or more characteristics associated with disease progression. The measurement data is collected for one or more locations within the lung. If several locations are measured within the lung, an evaluation parameter is derived from the measurement data related to the disease progression at the locations. The disease progression is then visualized in a geometrical representation of the lung, and suitable treatment is delivered at the visualized locations.

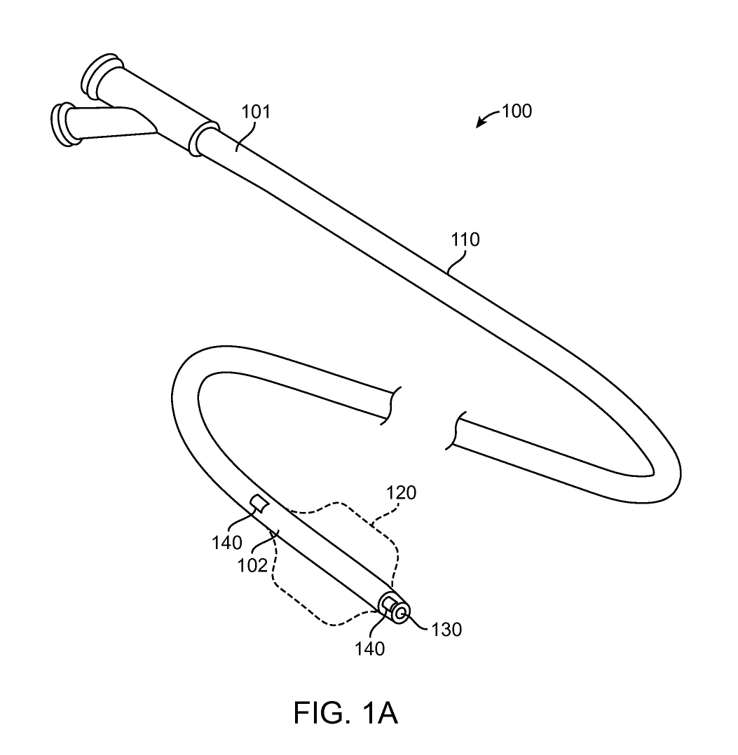

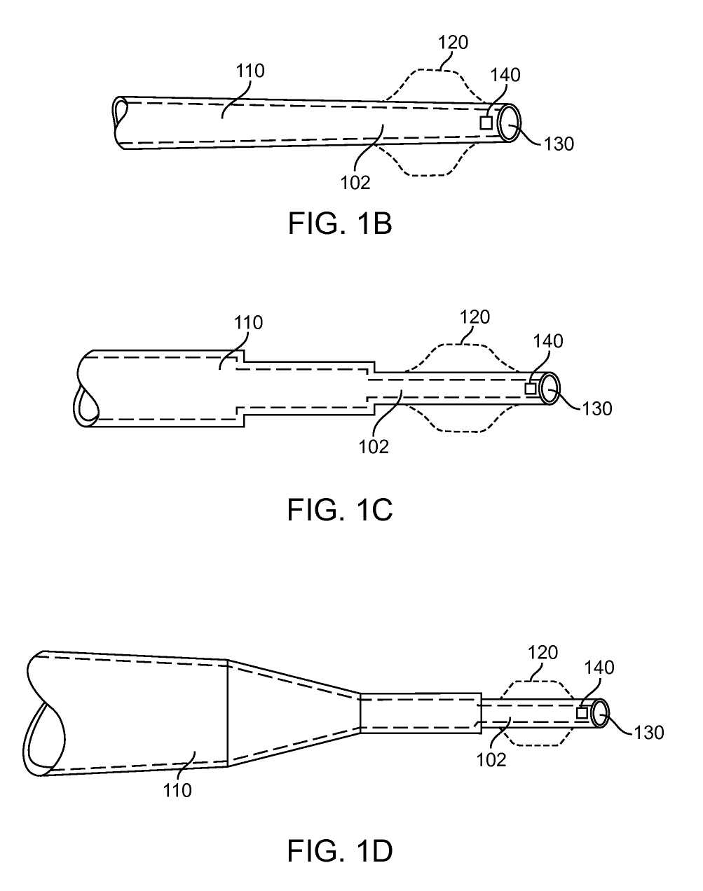

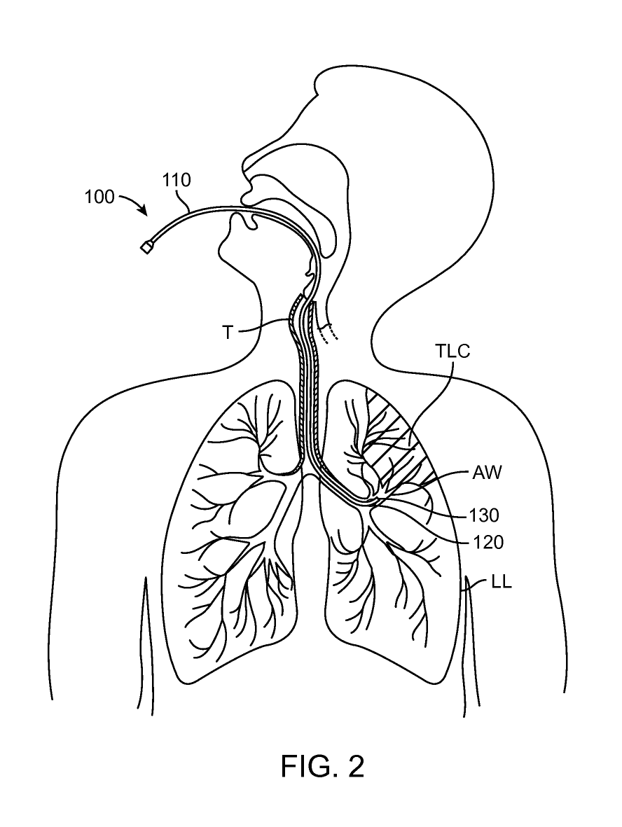

In each of the present embodiments, isolation of the lung comprises sealingly engaging a distal end of a catheter in an airway feeding a lung compartment, as shown in FIGS. 1A and 2. Such a catheter has been disclosed in co-pending published U.S. patent application Ser. No. 10/241,733, which is incorporated herein by reference. As shown in FIG. 1A, the catheter 100 comprises a catheter body 110, and an expandable occluding member 120 on the catheter body. The catheter body 110 has a distal end 102, a proximal end 101, and a lumen 130, (or alternatively multiple lumens), extending from a location at or near the distal end to a location at or near the proximal end. The proximal end of catheter 100 is configured to be coupled with an external control unit (not shown), and optionally comprises an inflation port (not shown). The distal end of catheter 100 is adapted to be advanced through a body passageway such as a lung airway. The expandable occluding member 120 is disposed near the distal end of the catheter body and is adapted to be expanded in the airway which feeds the target lung compartment. The lumen of the catheter may be cylindrical and of even diameter as shown in FIG. 1. In alternative embodiments shown in FIGS. 1B, 1C and 1D, the catheter lumen is configured to offer minimal resistance to airflow during exhalation and sampling. This is done so that the sampling process has a minimal effect on the flow or pressure characteristics being measured. Thus, in one embodiment of the catheter lumen shown in FIG. 1B, the diameter may gradually taper from being broader at the proximal to narrower at the distal end. In another embodiment shown in FIG. 1C, the diameter of the catheter may reduce in stages from being broader at the proximal portion to narrower at the distal end. In another embodiment shown in FIG. 1D, the catheter may have a combination of sections of varying degree of taper as well as of different uniform diameters. In the embodiment shown in FIG. 1D, for example, the distal-most portion of the catheter is of uniform diameter, which is configured to be held within a bronchoscope (not shown). Immediately proximal to that distal portion is a portion configured to engage with the valve of the bronchoscope. Thereafter, there is a slow transition to a third diameter as the catheter exits the bronchoscope.

In one aspect of the invention, catheter 100 is introduced into the target lung compartment TLC which is isolated by inflating the occlusion element 120. Thereafter, a physiological, anatomical or biological characteristic is assessed at the location in the TLC. For purposes of description, the measurements obtained by the catheter are described as being of the TLC. It should be understood, however, that such a description includes the TLC, the airway between catheter and TLC and any similar anatomy.

FIG. 2 shows an embodiment of a catheter configured to carry out the method described above. The catheter is configured to isolate the lung by having a distal portion that sealingly engages an airway feeding a lung compartment. Such a catheter has been disclosed in co-pending published U.S. Patent Application 2003/0051733, which is incorporated herein by reference. As shown in FIG. 2, the catheter 100 comprises a catheter body 110, and an expandable occluding member 120 on the catheter body. The catheter body 110 has a distal end 102, a proximal end 101, and at least one lumen 130, extending from a location at or near the distal end to a location at or near the proximal end. The proximal end of catheter 100 is configured to be coupled with an external console (not shown), and optionally comprises an inflation port (not shown). The distal end of catheter 100 is adapted to be advanced through a body passageway such as a lung airway. The expandable occluding member 120 is disposed near the distal end of the catheter body and is adapted to be expanded in the airway which feeds the target lung compartment. Additionally and optionally, catheter 100 further comprises at least one sensor 140 located within or in-line with the lumen 130 for sensing characteristics of various gases in the air communicated to and from the lung compartment. The sensors may comprise any suitable sensors or any combination of suitable sensors. Exemplary sensors include pressure sensors, temperature sensors, air flow sensors, gas-specific sensors, or other types of sensors. As shown in FIG. 2, the sensors 140 may be located near the distal end 102 of the catheter 100. Alternatively, the sensors 140 may be located at any one or more points along the catheter 100, or in-line with the catheter and within the console with one or more measuring components.

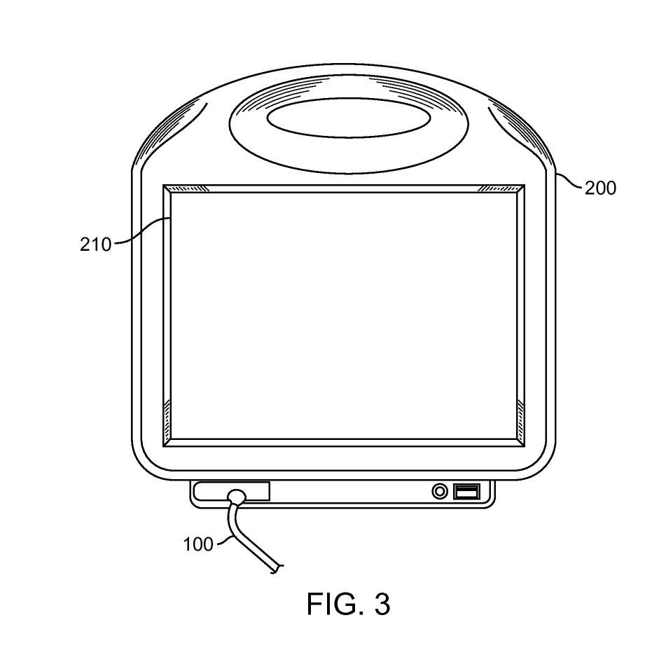

The proximal end of the catheter 100 is configured to be associated with a console 200, which is shown in FIG. 3. The console 200 comprises one or more measuring components (not shown) to measure lung functionality. The measuring components may take many forms and may perform a variety of functions. For example, the components may include a pulmonary mechanics unit, a physiological testing unit, a gas dilution unit, an imaging unit, a mapping unit, a treatment unit, or any other suitable measuring components. The components may be integral with or disposed within the console 200. Optionally, console 200 may also comprise mechanisms to introduce a gas or a mixture of gases from a gas dilution unit into the isolated lung compartment via one or more catheter lumens. The console 200 comprises an interface for receiving input from a user and a display screen 210. The display-screen 210 will optionally be a touch-sensitive screen, and may display preset values. Optionally, the user will input information into the console 200 via a touch-sensitive screen mechanism. Additionally and optionally, the console may be associated with external display devices such as printers, or chart recorders. The methods of the present invention will now be described with reference to the above embodiments.

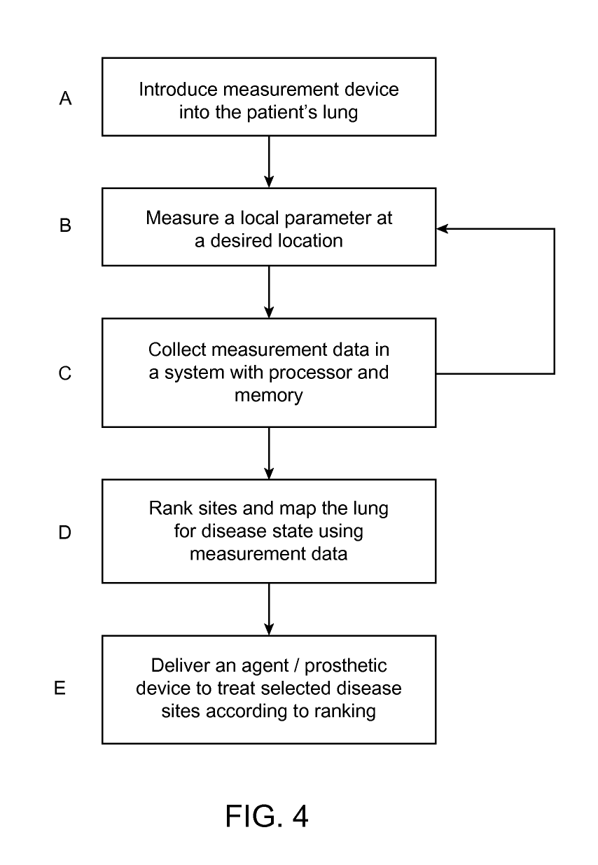

The various steps in one embodiment of the invention are illustrated in the schematic flow diagram shown in FIG. 4. As shown at Step A, a measurement device in the form of a catheter (with sensors within or arranged in line with the catheter) is inserted into the lung, and advanced to an assessment site. The type of data collected by the sensors may include anatomical, physiological, or biological information characterizing the disease state, and positional information to enable mapping and computerized rendering of the interior of the lung. The catheter is attached at its proximal end to a console and the measured data is collected by a data acquisition and analysis system attached to or contained within the console.

In step B, data or measurements of a local parameter (which includes anatomical, physiological or biological characteristics) are obtained from the assessment site. In step C, the characteristic data collected by the sensors, which relate to the state of disease progression at different locations within the lung, are collected along with the corresponding positional (anatomical) information. The information is collected within a database that is stored within a system with processor and memory attached or contained within the console. Steps B and C are repeated across a number of sites in the patient's lung as required. Thereafter, in step D, the collected data is then used to derive a score corresponding to each measurement site. The score may be a suitable function of the anatomical, physiological and biological characteristics measured and optionally may indicate an order of priority for treatment. A functional algorithm is used to derive the score and the algorithm may vary depending on the lung disease being treated. The scores, which are indicative of the severity of the disease at different locations within the lung, are then displayed on the console for viewing, for example in graphical form or as an anatomical representation.

Thereafter, the identified diseased portions may be treated as shown in step E. Treatment may be optimized by the aforementioned scoring, which may score the sites according to a feature such as a site's geometrical location or the state of disease progression. The disease may then be treated by delivering a therapeutic agent at one of the assessment sites. Alternatively, the lung compartment may be treated by deploying a device such as a flow restrictor at the airway location.

An exemplary physiological characteristic is the presence and/or degree of collateral ventilation which can be measured using any of the methods disclosed in copending U.S. Patent Applications 2003/0051733 and 2006/0264772. An exemplary biological characteristic is the presence of a gas such as nitric oxide, which is often found in diseased lung segments. An exemplary physiological characteristic is the presence of an air leak, which may also be determined by measurements of collateral ventilation.

In another aspect of the present invention, the locations or positions of the assessment sites are recorded or tracked. The locations of the sites are thereafter mapped into a computerized database located within the system attached to console 200. The data measured by sensors 140 and the position data are then used to calculate a ranking parameter for prioritizing treatment. The ranking parameter is obtained using an algorithm based on the determined number of diseased regions in the lung, one or more of the physiological, anatomical or biological characteristics of each site, and proximity of one or more assessment sites to at least one of the diseased regions.

Another aspect the invention involves determining a treatment plan based on the ranking of various sites within the lung and the disease state of the patient. The treatment plan may include determining which sites are to be treated first based on anatomical location or the progression of the disease. Thereafter, the assessment site may be treated in a number of ways using the treatment plan. The specifics of the treatment may depend upon the disease and may include installation of flow control elements such as a plug, a one-way valve, a two-way valve, or a two-way valve fitted with a drug depot. Alternatively, minimally invasive surgical sealing of the airway or surgical lung volume reduction may be practiced. Additionally and optionally, treatment may further include delivering a therapeutic agent to the TLC. The therapeutic agent can be in solid, liquid, gel or vapor form, and may be administered according to a treatment plan.

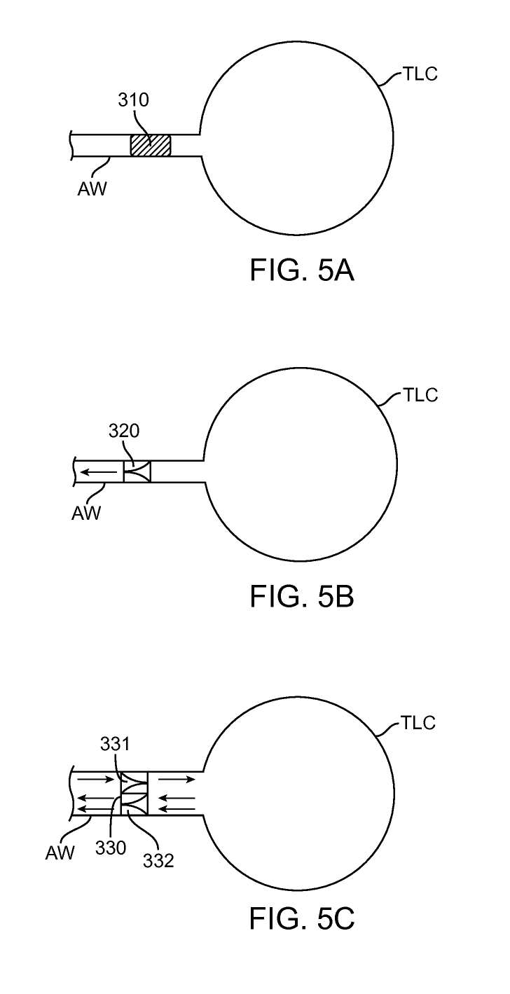

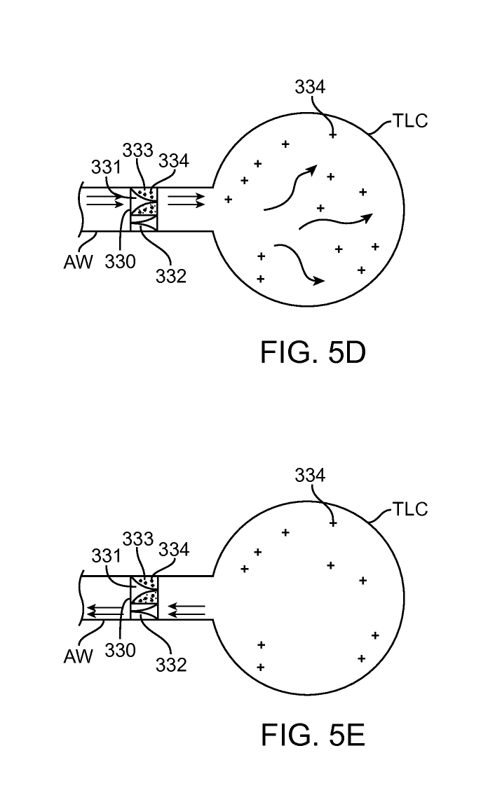

In one embodiment, if the degree of collateral ventilation is small or negligible in a patient with COPD such as emphysema, the treatment may involve lung volume reduction as shown in FIGS. 5A and 5B. In FIG. 5A, a plug 310 is installed at an airway AW leading to the target lung compartment. The plug may be installed by implanting at the location a swellable material such as collagen hydrogel that occludes the airway by absorbing water. Alternatively, the plugging is achieved by releasing a substance in liquid or gel form, which subsequently hardens. The substance can be a biocompatible polymer or adhesive, for example. Plugging the target lung compartment would prevent further inflation of the TLC and enable the trapped air to diffuse through capillary circulation.

Alternatively, as shown in FIG. 5B, a one-way valve 320 that permits only expulsion of air from the TLC may be installed at the airway location. The one-way flow control element would enable gradual evacuation of the affected lung portion by progressively reducing the amount of residual air in the isolated lung portion and preventing reinflation.

In alternative embodiments, the airway can be surgically sealed by suturing, for example. The sealing may be accompanied by active methods of lung volume reduction such as endobronchial aspiration or externally forcing air out of the TLC through surgical means.

In another embodiment, an airway bypass may be produced by creating an artificial opening between the affected portion of lung and the healthy portion to effect lung volume reduction. The airway bypass may be provided by installing a one-way flow control element across the bronchial wall.

In another embodiment of the invention shown in FIG. 5C, a two-way flow control element may be installed, if it is desired according to the treatment plan that a controlled two-way exchange of air is to be maintained at a selected location in an airway. As shown in the figure, two-way flow control element 330 is installed in an airway AW, comprising flow control portion 331 allowing inhalation and flow control portion 332 permitting exhalation from the TLC.

In another embodiment shown in FIGS. 5D and 5E, flow control portion 331 allowing inhalation is provided with drug depot 333 containing adequate dosage of drug 334 to serve to treat a lung disease. During inhalation, flow control element 331 permits drug particles 334 to be entrained in the inhaled air and reach target locations within the TLC, as shown in FIG. 5C. Drug particles 334 may be partially absorbed at diseased locations. During exhalation, flow control element 332 may facilitate lung volume reduction. Although some of the inhaled drug particles may escape through flow control element 332, the drug is targeted to the area it is most needed. General systemic exposure to the drug would thus be limited, thereby minimizing side effects. Examples of drugs that may be administered in this manner include steroids or anticholinergics.

The use of drugs may be particularly useful in treatment of diseases such as lung cancer, wherein general systemic exposure to the agent may be undesirable, while high concentrations may be required to be delivered on a sustained basis to the disease location. The method of the present invention as disclosed in FIG. 5C may be used to treat in a controlled manner, cancerous growths or other lung disease requiring interventions using an anti-cancer chemotherapeutic agent.

In another embodiment, the invention may be used for the treatment of asthma. The endobronchial catheter 100 shown in FIG. 2 may be fitted with a sensor 140 for sensing the concentration of nitric oxide in tissue, which is generally indicative of inflammation accompanying asthma. Thereafter, a number of such locations requiring treatment are identified to arrive at a treatment plan, as described in previous embodiments. To treat asthma, a thermally controlled heating element is deployed at the distal end of catheter 100. The heating element is deployed to be in contact with the airway walls and controlled thermal heat is applied for an appropriate period of time to effect inactivation of the airway muscles causing asthma. Alternatively, the treatment may comprise the release of a drug into the TLC via the catheter 100. The drug may be in solid, liquid, gel or vapor form and may include one or more of bronchodilators, steroids and anticholinergics.

Another treatment option is the use of a tissue prosthesis or a chest tube to treat a bronchopleural leak. The tissue prosthesis may be made of any suitable biosorbable material.

In all the above embodiments the efficacy of treatment may be confirmed by introducing an assessment catheter to confirm the reduction of a disease marker parameter. For example, the catheter may be used to determine if any air leaks exist post-procedure so that subsequent treatment options may be assessed. Further, the catheter can be used to quantify the effectiveness of drug therapy, valve placement or the sealing agent at the local level.

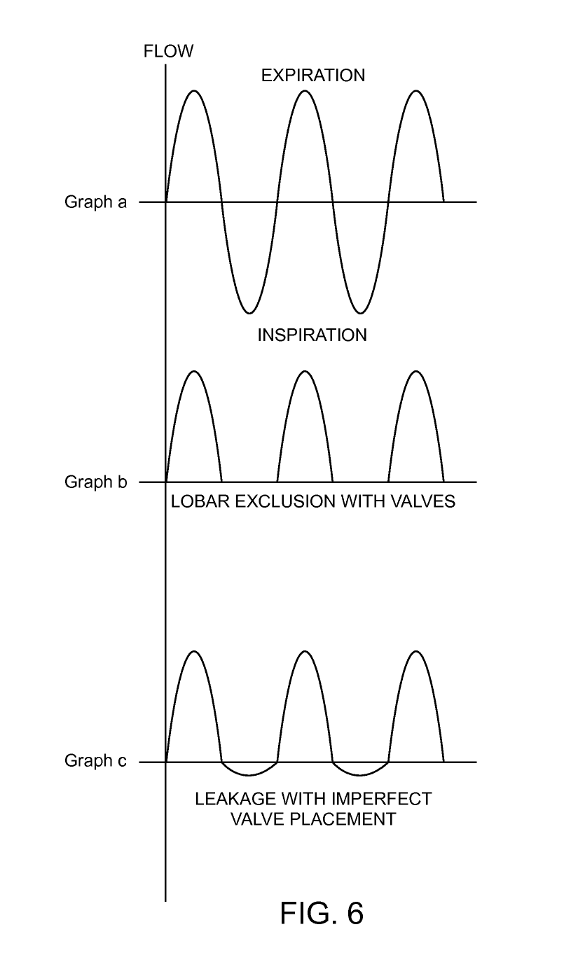

The particular example of using the catheter to determine the effectiveness of valves or other implants designed to induce ELVR is shown in FIG. 6. Normal respiration is shown in Graph a of FIG. 6, and exhibits equivalent flow in both expiration and inspiration phases. If the lobe has been implanted with one-way valves, there should be no detectable inspiratory flow. Thus, the catheter and console would detect airflow that exhibits the characteristics shown in Graph b of FIG. 6, where flow is only present in the expiratory direction. However, if the valves are ineffectual or compromised allowing air to ‘leak’ due to placement or other anatomical limitations, some inspiratory flow would still be present. This is exemplarily shown in Graph c of FIG. 6.

It should be noted that the above example can also be used to determine the presence of physiological air leaks occurring in an untreated lung as a diagnostic tool prior to any treatment at all. The same process is used, and a similar graph to Graph c would be obtained if the lung compartment contained any inherent air leaks.

While the above is a complete description of various embodiments, various alternatives, modifications, and equivalents may be used. Therefore, the above description should not be taken as limiting the scope of the invention, which is defined by the appended claims.

What is claimed is:

1. A method for selecting one or more treatment sites in a diseased lung, the method comprising:
introducing an assessment catheter into an airway leading to a first assessment site in the lung;
expanding an occluding member on the catheter to form a seal with an inner wall of the airway and thus isolate the first assessment site;
measuring at least one physiological, anatomical or biological characteristic of the first assessment site using the catheter;
calculating a score for the first assessment site based on the measured characteristic and a predetermined algorithm;
repeating the steps for at least a second assessment site in the lung; and
selecting at least one treatment site based on the scores of the assessment sites.

2. The method of claim 1, further comprising repeating the steps for at least a third assessment site.

3. The method of claim 1, wherein at least the calculating step is performed by a console coupled with a proximal end of the catheter, and wherein the scores are displayed on the console.

4. The method of claim 1, wherein the physiological characteristic is collateral ventilation.

5. The method of claim 4, wherein the collateral ventilation is assessed to treat an air leak.

6. The method of claim 1, wherein the biological characteristic is nitric oxide.

7. The method of claim 1, wherein the anatomical characteristic is an air leak.

8. The method of claim 1, wherein the algorithm is based on a determined number of diseased regions in the lung, the at least one physiological, anatomical or biological characteristic of each site, and proximity of each assessment site to at least one of the diseased regions.

9. The method of claim 1, further comprising treating the treatment site.

10. The method of claim 9, further comprising introducing an assessment catheter into the lung to confirm efficacy of treatment.

11. The method of claim 9, wherein treating comprises implanting a one-way flow control element into an airway leading to a portion of the lung afflicted by emphysema.

12. The method of claim 11, wherein the flow control element is selected from the group consisting of a plug, a one-way valve and a two way valve.

13. The method of claim 11, wherein the flow control element is provided with a drug depot configured to provide sustained release of a drug.

14. The method of claim 13, wherein the drug depot is configured to release at least one of: steroids and anticholinergics.

15. The method of claim 9, wherein treating comprises performing endoscopic lung volume reduction.

16. The method of claim 9, wherein treating comprises introducing a drug into the treatment site through a treatment catheter.

17. The method of claim 9, wherein treating comprises performing bronchial thermoplasty.

18. The method of claim 9, wherein treating comprises installation of a chest tube.

19. A method for assessing the effectiveness of a treatment, the method comprising:
identifying an airway that has been occluded with a one-way valve, wherein the one-way valve is configured to allow expiration but limit inhalation;
introducing a catheter into the identified airway, the catheter comprising a distal end, a proximal end, and a lumen therebetween, wherein the distal end comprises an expandable occluding element configured to sealingly engage the airway, wherein the proximal end comprises an inflation port to expand the occluding element, and wherein the lumen is in-line with at least one sensor for measuring a respiratory characteristic; and
measuring flow through the airway to determine whether flow exists during inhalation, wherein the presence of flow indicates ineffective valve placement.

20. The method of claim 19, further comprising measuring pressure during inhalation, wherein the presence of pressure indicates ineffective valve placement.
